(19) United States
(12) Patent Application Publication
(10) Pub. No.: US 2010/0236959 A1
(43) Pub. Date: Sep. 23, 2010

(54) Absorbent Article and Test Article Combination

(75) Inventors: John FERRER, Mason, OH (US); Dean Larry DU VAL, Lebanon, OH (US); René STAEBE, Schwalbach (DE)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 12/723,800

(22) Filed: Mar. 15, 2010

Related U.S. Application Data
(60) Provisional application No. 61/160,782, filed on Mar. 17, 2009.

(51) Int. Cl. A61B 19/00

(52) U.S. Cl. 206/440

(57) ABSTRACT

A combination of an infant article for use by an infant, having an external surface, comprising a first material, for example a backsheet material with a testable property, such as stretch or softness, the infant article being comprises in a packaging material; and a separate first test article, for testing by a care-taker and for subsequent use by an infant, the test article having an external surface comprising the first material with the testable property. The test article is for example curved and/or three-dimensional, having an internal volume comprising a resiliently compressible material, such as a soft infant toy or pillow.

### CROSS REFERENCE TO RELATED APPLICATIONS

This application claims the benefit of U.S. Provisional Application No. 61/160,782 filed on Mar. 17, 2009, which is herein incorporated by reference in its entirety.

### FIELD OF THE INVENTION

The present disclosure relates to a combination of an article for an infant, such as a disposable absorbent article and a separate test article for testing by a potential buyer, wherein each include a first material with a user-testable property, such as for example softness or stretch. The test article may be three-dimensional, having an internal volume including in one embodiment herein, a resiliently compressible material.

### BACKGROUND TO THE INVENTION

Producers of consumer goods often face the problem how to communicate to the purchaser of the goods certain features of the goods. For certain products and in certain instances, product samples can be provided to the purchaser in store that can be tried immediately to facilitate the purchaser's choice, such as for perfumes.

In other instances, the actual product can be tried in store. However, for products that need to remain in their packing material, such as hygiene articles, such trial of these new or beneficial product features in store is not possible.

Furthermore, for certain products, such as infant products, the purchaser of the product is not the end user (e.g., wearer) of the product and thus not the person benefitting from these new of beneficial product features. It can then be difficult to understand/experience a product, even more so if the end user is an infant (baby) that cannot communicate anything about the performance experience.

Thus, the producers of such goods face additional issues with the trial of such features and with communicating the benefits of such products.

Various solutions thereto have been proposed and used by the producers of such goods, including sample products being given out in store, or by mail. WO05/061338 suggests attaching a sample swatch for sensory touch to the external surface of the packaging of an absorbent article, which can be tried without opening the packaging.

It has been found that touching or examining such a packaging-swatch of a wearable absorbent product does not provide the right sensation or observation, or does not provide a realistic sensation or observation for the user. In use, such an article is applied against the body of the wearer and may provide a different sensation or performance compared to a swatch attached to a packaging material (if comparable at all).

Furthermore, it has been found that certain properties such as softness are not always easy to test by touching and feeling it by hand. In some instances, purchasers often tend to use the skin of the cheek to test the softness of an article.

Furthermore, other performance properties may have to be communicated to the purchaser in a way that is directly comparable with the in-use performance, and that can not be communicated by a packaging-swatch that is attached to a container.

Thus, there is a need to provide an improved method for testing a material property by a purchaser or to better experience or understand the article he or she may be purchasing, in particular in the case of infant articles that are ultimately not used by the purchaser, e.g., the test method closer resembling the real in-use experience.

As one solution thereto, it has been found that it is beneficial to provide with the infant article a further test article, that also includes the material of the article that is to be tested, the test article being suitable for in store testing by the purchaser and being separate from the infant article, and whereby the test article can subsequently be used by the user (infant) of the actual article. Furthermore, it has been found as improved method to let the purchaser test an article, that the additional, separate test article should resemble the wearer's (user's) curved shape and/or body-like resiliency/compressibility, so that it may be beneficial that the test article is three dimensional and/or having a certain resiliency/compressibility, like the body of a wearer (user) would have.

Testing such a three dimensional test article (for example by touch) provides a more realistic sensation or performance experience for the purchaser, which can be easier translated into an expected performance or sensation of the actual article.

### SUMMARY OF THE INVENTION

The present disclosure relates to a combination of:
a) an infant article for use by an infant, having an external surface, including a first material (1) with a testable property, the infant article being included in a packaging material; and
b) a separate first test article for testing by a care-taker and for subsequent use by an infant, the test article having an external surface including the first material (1) with the testable property.

The infant article is for example a disposable absorbent hygiene article, for example an infant diaper.

In one embodiment, the test article is three-dimensional, having an internal volume including a resiliently compressible material, such as pulp, cotton, and further materials described herein.

The test article may be infant clothing, infant bedding, infant pillow or infant toy, such as an animal or doll.

In one embodiment, the user-testable property is the softness sensation and/or the flexibility and/or the stretchability of the first material (1).

The first material (1) may be a nonwoven material, as further described herein below, and the test article may have an external surface (partially) made of this first material (1).

In one embodiment, because the current test article may be resiliently compressible and because it may be an infant toy, the user is more inclined to place it immediately against the cheek for testing its property, e.g., softness. Thus, the present test article provides improved instinctive performance testing.

In another embodiment herein, a combination whereby the infant article includes a two-dimensional symbol, such as an icon, and the test article is the icon in a three-dimensional form.

### FIGURES

FIG. 1 shows a view of the back of a diaper useful herein, showing the surface that is in use facing the wearer's cloths, the backsheet (2) including the first material (1).

FIG. 2 shows a view of a test article useful herein, including also the first material (1) shown in FIG. 1.

FIG. 3 shows an alternative test article useful herein.

FIG. 4 shows an alternative test article useful herein.

### DETAILED DESCRIPTION

Herein, “comprise” and “include” mean that other elements and/or other steps which do not affect the end result can be added. Each of these terms encompasses the terms “consisting of” and “consisting essentially of”.

Herein, “body facing surface” refers to surfaces of absorbent articles and/or their component materials which face the body of the wearer, while “garment facing surface” refers to the opposite surfaces of the absorbent articles and/or their component materials that face away from the wearer when the absorbent articles are worn. Absorbent articles herein (and components thereof, including the topsheet, backsheet, absorbent core, and any individual layers of their component materials) typically have a body facing surface and a garment facing surface.

As used herein “absorbent article” refers to devices which are intended to be placed against the skin of a wearer to absorb and contain the various exudates discharged from the body. Examples of absorbent articles include adult incontinence articles such as pads, briefs and diapers; infant (i.e., baby, toddler, child) diapers, including diapers with fasteners, pre-fastened diapers, pant-like diapers such as training pants; diaper holders. Further examples of absorbent articles are feminine hygiene articles such as sanitary napkins and panty-liners. In one embodiment, the absorbent articles are infant diapers as mentioned above.

As used herein “disposable” is used to describe absorbent articles for single use, which are not intended to be laundered, restored or otherwise reused as an absorbent article after a single use.

As used herein “resiliently compressible” means that the test article or material of its internal volume is at least partially compressible by a force into a compressed state but does not maintain the compressed state once the force is removed; typically it means that the test article or internal volume thereof, or part thereof is compressible by a force (e.g., by a force that can be applied by an adult by a closing hand movement) into a compressed state, and recovers at least partially after removal of the force (i.e., at least 50%, so that its volume increases after release of the force with at least 50% of the decrease in volume caused by the force, within one minute, at 20° C., 50% humidity).

### Disposable Absorbent Hygiene Article

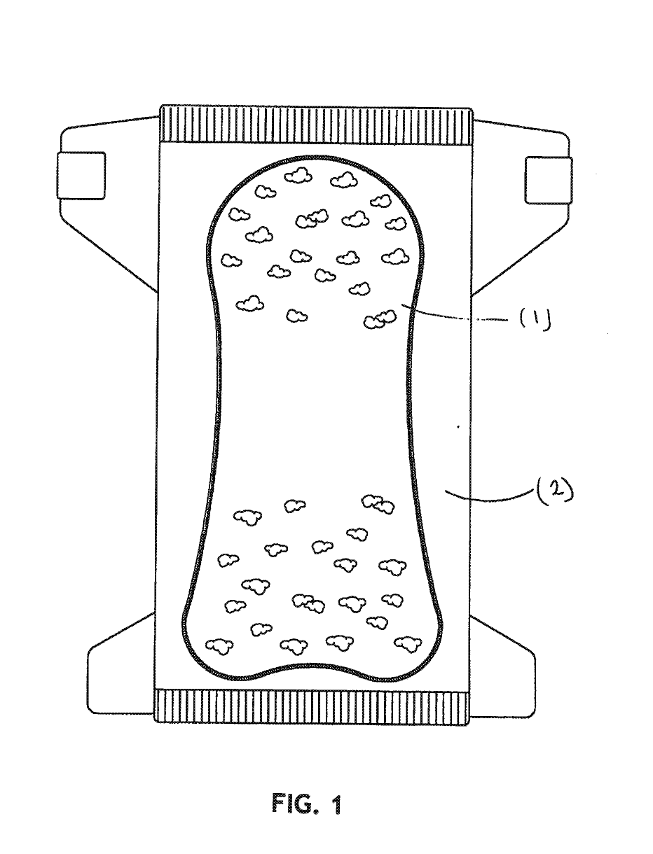

The absorbent hygiene article herein is to be worn against the skin of a user, and it is typically an adult incontinence article, infant diaper or sanitary napkin or panty-liner, and in an embodiment herein, the article is an infant diaper, including diapers with fasteners, as for example shown in FIG. 1, and pants-type diapers.

Suitable diapers are disclosed in, e.g., U.S. Pat. No. 3,860,003 issued to Buell on Jan. 14, 1975; U.S. Pat. No. 5,151,092 issued to Buell et al. on Sep. 29, 1992; U.S. Pat. No. 5,221,274 issued to Buell et al. on Jun. 22, 1993; and U.S. Pat. No. 5,554,145 issued to Roe et al. on Sep. 10, 1996. As used herein “incontinence article” refers to pads, undergarments, inserts for absorbent articles, capacity boosters for absorbent articles, briefs, bed pads, and the like regardless of whether they are worn by adults or other incontinent persons. Suitable incontinence articles are disclosed in, e.g., U.S. Pat. No. 4,253,461 issued to Strickland, et al. on Mar. 3, 1981; U.S. Pat. Nos. 4,597,760 and 4,597,761 issued to Buell; the above-mentioned mentioned U.S. Pat. No. 4,704,115; U.S. Pat. No. 4,909,802 issued to Ahr, et al.; U.S. Pat. No. 4,964,860 issued to Gipson, et al. on Oct. 23, 1990; and PCT Publication No. WO 92/11830 published by Noel, et al. on Jul. 23, 1992. As used herein “pant-like diaper” refers to a specific diaper having fixed sides and leg openings. Pant-like diapers are placed in position on the wearer by inserting the wearer's legs into the leg openings and sliding the pant-like diaper into position about the wearer's lower torso. Suitable pant-like diapers are disclosed in, e.g., U.S. Pat. No. 5,246,433, issued to Hasse, et al. on Sep. 21, 1993.

The absorbent article herein has an inner, body facing surface and an outer, garment facing surface opposed to the inner surface.

The chassis of the absorbent article includes a topsheet, which may be liquid pervious, and a backsheet, that may be liquid impervious. The chassis encloses an absorbent core. The chassis has a periphery which is defined by the transverse outer edges of the chassis with longitudinal edges and end edges.

The backsheet may be a liquid impervious backsheet, as known in the art. The backsheet (2) may include the first material (1) described herein, as for example shown in FIG. 1. An example backsheet (2) herein includes a laminate sheet of a nonwoven layer and a film layer or a film-coated nonwoven layer, whereby in either case the backsheet (2) or the nonwoven layer may be, or may include, the first material (1) described herein. Hereby, the film layer or film coating faces the absorbent core of the article; the nonwoven layer may form part of) the external surface of article, and may thus for example face the wearer's cloths in use. FIG. 1 shows such an article including a backsheet (2) that includes a nonwoven layer on its external surface, which is the first material (1) as used herein; or the backsheet (2) includes a laminate layer of a film and a nonwoven, or includes a film-coated nonwoven layer, and the layer is the first material (1), as used herein.

The topsheet is may be compliant, soft feeling, and non-irritating to the wearer's skin. The topsheet may include or be the first material (1) described herein.

The topsheet may be liquid pervious permitting liquids (e.g., urine) to readily penetrate through its thickness.

The absorbent article herein may have one or more pairs of (elasticated) cuffs, including so-called leg cuffs and barrier cuffs and anal and/or vaginal cuffs, which provide improved containment of liquids and other body exudates. The cuffs of a pair may be mirror images of one another in the longitudinal axis of the article and they are opposing one another, whereby a cuff or panel is positioned on either longitudinal side of the core and/or of the chassis. Suitable cuffs are described in for example U.S. Pat. No. 3,860,003; U.S. Pat. Nos. 4,808,178 and 4,909,802; U.S. Pat. Nos. 4,695,278 and 4,795,454.

Such elasticated cuffs, including side panels, may also be made of or include the first material (1), as described herein.

The cuffs and/or topsheet may include an active ingredient, like a skin care lotion.

The article may also include, one or two pairs of opposing, side panels, also referred to as “ears”, each of the side panels being attached to or in close proximity to either longitudinal side edge of the backsheet (2) or absorbent core or topsheet, or part of They may be positioned such that they extend outwards from the backsheet (2) or core or topsheet. It may be that the article has a pair of opposing side panels in the front portion of the article and a pair of opposing side panels in the back portion of the article. The side panels of a pair may be mirror images of one another in the Y-axis of the article. The side panels may also include or be made of the first material (1) herein.

The absorbent core generally is disposed between the topsheet and the backsheet (2). The absorbent core may include any absorbent material that is generally compressible, conformable, non-irritating to the wearer's skin, and capable of absorbing and retaining liquids such as urine and other certain body exudates.

The absorbent core may include one or more materials selected from a wide variety of liquid-absorbent materials commonly used in disposable diapers and other absorbent articles such as comminuted wood pulp which is generally referred to as airfelt. Examples of other suitable absorbent materials include creped cellulose wadding, meltblown polymers including coform, cross-linked cellulosic fibers, tissue including tissue wraps and tissue laminates, absorbent foams, absorbent sponges, superabsorbent polymers, absorbent gelling materials, or any equivalent material or combinations of materials. The configuration and construction of the absorbent core may also be varied, e.g., the absorbent core may have varying caliper zones, a hydrophilic gradient, a superabsorbent gradient, or lower average density and lower average basis weight acquisition zones; or may include one or more layers or structures.

Further, the diaper or incontinence articles may include a fastening system, which may include or be the first material (1) herein. Fastening systems may include fastening tabs and landing zones, wherein the fastening tabs are attached or joined to the back region of the article, for example attached to the back region side panels (or ears) and the landing zones are part of the front region of the diaper.

### First Three-Dimensional Test Article

The test article herein includes also the first material (1), as described herein, so it can be tested by a purchaser. It is separate from the actual article, which means it is not packed with the article or attached to the article, i.e., so it can be tested separate from the article. The test article may be suitable for subsequent use by an infant.

The test article may be a three-dimensional test article, with an internal volume. The internal volume may be at least 4 cm³, or at least 10 cm³, or at least 15 cm³. In one embodiment herein, the article is resiliently compressible, and may have a resiliently compressible material in its internal volume.

The test article herein includes a first material (1) that has a certain testable property, as described herein below, and that first material (1) is also included by the absorbent article. The test article may include this material on its surface, or on part of its surface. The test article may also include additional material that may not be included by the absorbent article; the first material (1) may then be either attached to other components of the test article and/or being superposed onto this other material.

The test article herein may have at least one curved surface including the first material (1). Thereby, a user can test the first material (1) on a curved surface that more accurately resembles the real-use performance or property of the first material (1).

The test article may have an internal volume, including a material that provides the compressibility and resiliency to the article, i.e., a material than may be compressed by a force but recovers at least partially after removal of the compression force; the internal volume may include: a gas enclosed by a flexible gas-impermeable material, e.g., air; or a liquid in a flexible liquid-permeable material; it may include solid compressible and resilient materials, like fillers know in the art for children's toys, including cotton, polyester, plastic beads, pellets, sand, feathers, foam, fibers, or combinations thereof. Examples of other fillers include absorbent materials used in absorbent articles, including cellulose wadding, tissue, absorbent foams, absorbent sponges, or any of the absorbent materials described herein above. Also any combinations of the materials above may be used.

Such internal volume material may be enclosed and thereby contained by the first material (1) and/or by an additional cover material, which may be a flexible sheet or layered material, such as a woven or nonwoven material or film material. The additional cover material may then be (partially) covered the first material (1), so at least part of the external surface of the test article includes the first material (1).

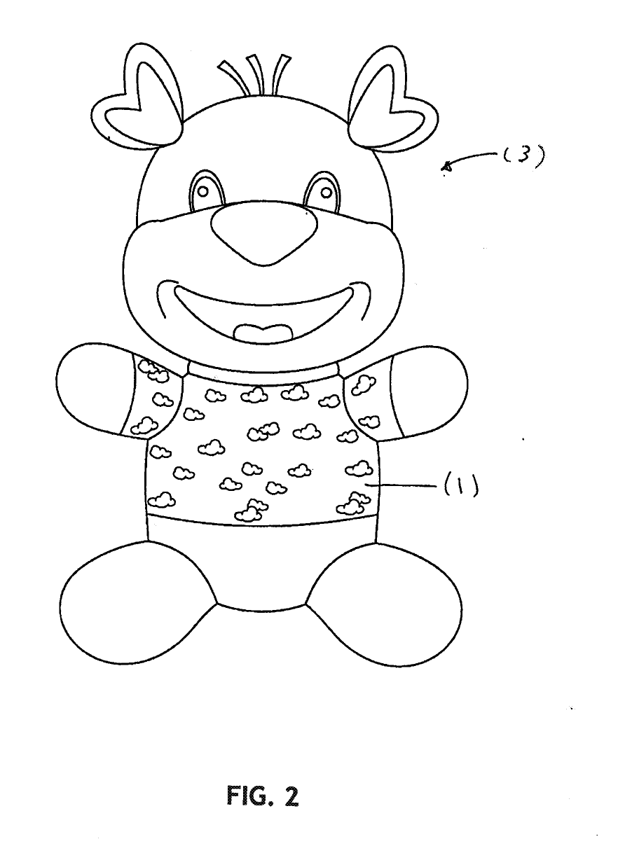

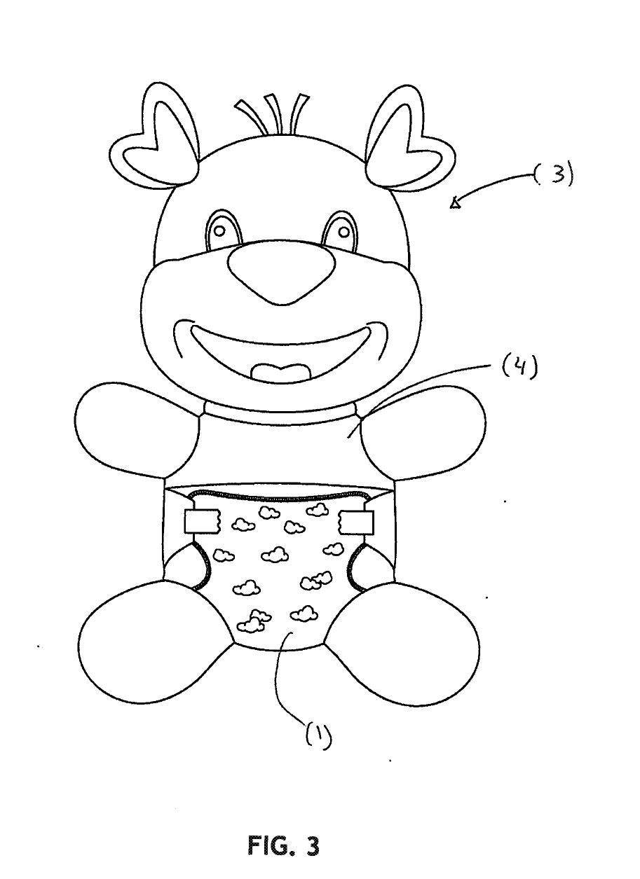

An example test article is an infant (baby, child) toy, including a ball, a doll, or toy animal. Examples of such a toy are shown in FIGS. 2 and 3. In one embodiment, the toy's outer-surface may consist of the first material (1). In another embodiment, the test article, e.g., toy, may only have a selected surface area including the first material (1), such as a doll or animal (3) wearing a absorbent article, as shown in FIG. 3, or clothing, as shown in FIG. 2, and the absorbent article or clothing of the toy including or being made of the first material (1). The absorbent article worn by the toy (3) or the clothing of the toy (3), made of the first material (1) is in one embodiment an integral part of the toy (3), as shown in the FIGS. 2 and 3, that then maybe be attached to a further material (4) to form together the surface of the toy (3).

The first material (1) may be permanently present on the test article, so that the article readily testable by a user, without activation, e.g., without opening a packaging material, lifting a covering material etc.

Such (toy) test articles herein may be made by know techniques, including for example shaping an outer surface area, including the first material (1), into a three dimensional shape, or attaching variously shaped pieces of material, (whereof at least one including the first material (1)) together to form the three dimensional shape, by for example sewing, stitching, stapling; and then internal stuffing of the three dimensional shape with a filler material, as for example described above.

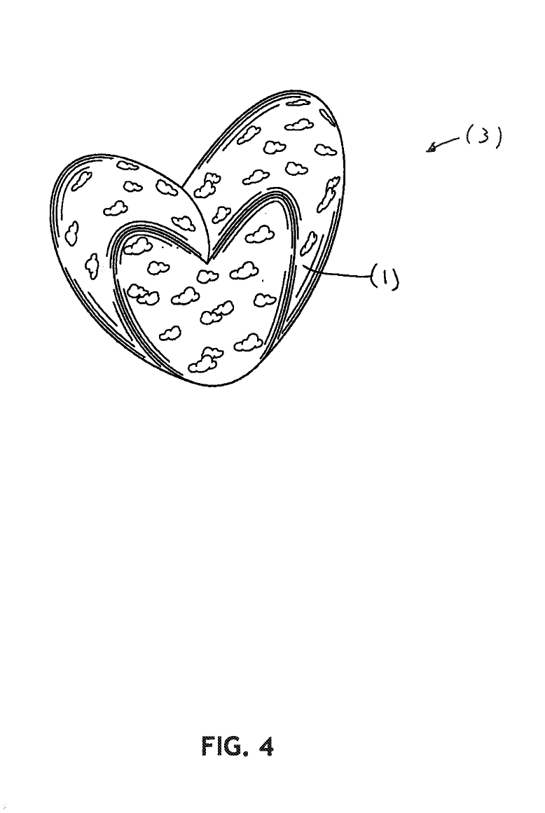

In one embodiment, the first material (1) of the test article has a color, colored pattern, printed pattern, graphics or text which is substantially the same as the color, colored pattern, printed pattern, graphics or text of the first material (1) of the absorbent article, as shown in FIGS. 1 and 2 and 3 and FIG. 4, which exemplified a first material (1) with a printed pattern of colored clouds.

The test article may also, or alternatively, include graphics or text, related to the testable property.

The toy test article may have optional additional component, such as button eyes and yarn hair.

The test article may have a surface area where the first material (1) is not present. In one embodiment, to enhance the soft feeling of the outer surface including the test material, the test article includes one or more layers of additional padding material under part of the outer surface where the test material is present. The padding may for example be a compressible woven fabric or a polymeric foam, e.g., latex).

In one embodiment, the test article's outer surface also includes a second material with a user-testable property, to that the user can compare this second material with the first material (1), or more precisely, to compare the testable property of the second material and the testable property of the first material (1). The second material may also be included by a curved surface of the test article.

In the latter embodiment, the combination of the absorbent article and the test article may include also instructions for the user (tester/purchaser) how to test and compare the first material (1) and second material, described in more detail below.

### First Material (1) and User-Testable Properties Thereof

The user-testable property is readily testable by visual inspection, auditory inspection, or may be by touch. Examples of such properties that are testable by visual inspection include the surface structure or pattern of the first material (1); which, in one embodiment, may be better represented on a curved or resiliently compressible article, that represent better the in-use situation. Examples of properties that are testable by auditory inspection, is the fastening performance of the fasteners described above.

In one embodiment, the user-testable property is a property that may be inspected by touch, including the softness sensation and/or the stretchability and/or the flexibility of the first material (1), measurable by touch of the user (tester/purchaser).

In one embodiment herein, the first material (1) includes or is a nonwoven material, i.e., a nonwoven layer. This includes in particular hydro-entangled nonwovens, such as for example described in EP1684972A, carded nonwovens, calendar bonded nonwovens, embossed nonwovens, but it may also include spunbond nonwovens and meltblown nonwovens, or nonwovens made by any combination of method above, for example a spunbond nonwoven or meltblown nonwoven or carded nonwoven are subsequently embossed or further calendar bonded. The first material (1) may also be a combination of such nonwoven layers, such as a laminate material of two or more of any of the nonwoven layers described above.

The first material (1) may also include a film layer, and may be in addition to one of the nonwoven (laminate) layers described above.

For softness properties may be first material (1)s that include at least a hydro-entangled nonwovens, carded nonwovens, calendar bonded nonwovens and/or embossed nonwovens.

The nonwoven may include or be made of natural fibers (e.g., wood or cotton fibers), synthetic fibers (e.g., polymeric fibers, including polyolefins fibers, for example polypropylene and/or polyethylene fibers; polyester fibers) or from a combination of natural and synthetic fibers, or a combination of different natural fibers or a combination of different synthetic fibers. The fibers or filaments may also be made of two or more polymers, including polyolefins, including multi component or bicomponent fibers, may have a sheath/core arrangement. In one embodiment, the first material (1) may include multi-component fibers (filaments), or crimped multicomponent fibers (filaments), bi-component fibers. It may be a calendar bonded nonwoven including multicomponent fibers or bicomponent fibers, as for example described in EP1379718A.

The first material (1), e.g., the nonwovens or the fibers thereof, may include an additive, such as a softness enhancing additive or a skin care lotion.

In one embodiment, the first material (1) is a calendar bonded nonwoven material, for example having a bond pattern that is visible and/or that is distinguishable by touch. It may have a bond pattern having a geometrically repeating pattern of bonded areas, and a pattern of unbonded areas. Such bonded and unbonded areas may for example, each independently, have a surface area of from 0.01 cm² to 1 cm², or from 0.05 cm² to 0.5 cm². The bonded areas may cover for example from about 3% to about 50% of the surface of the first material (1).

The first material (1) may also be an embossed material, e.g., nonwoven, that may have an embossed pattern of densified areas (e.g., of lower loft) and low density areas (e.g., of higher loft). Thus, in one embodiment the first material (1) is a nonwoven material with raised areas and compressed regions, the raised areas having a lower fiber density relative to the compressed areas. Examples are described in U.S. Pat. No. 4,323,068 and EP907342A.

The first material (1) may have any basis weight, but in one embodiment, the first material (1) is or includes a nonwoven material with a basis weight from 13 gsm to 34 gsm.

In another embodiment herein, the first material (1) may also be or include an elastic material, including an elastic film, elastic nonwoven, or an elasticated film or nonwoven. This includes the embodiment that the first material (1) may include a non-elastic film or nonwoven, whereto one or more elastic materials, e.g., strands, are attached. Any of the above nonwovens may then be used and elasticated with elastic materials known in the art.

The first material (1) may also include so-called loops or hooks, commonly used in fastening materials.

### Further (Second) Materials and Further (Second) Test Articles

The test article may include a second material on its surface that is testable by a user (purchaser) in the same manner, e.g., by touch, as the first material (1).

Alternatively, the combination of the absorbent article and first test article includes an addition second test article, including such a second material, with a testable property

The property of the second material is of a different to the property of the first material (1), e.g., the same property may be tested, but the second material's property is of a different quantity or magnitude to the property of the first material (1). For example, the first material (1) is softer, or more flexible or more compressible, or more extendible than the second material; for example, the first and second material include both a nonwoven material, but the first material (1) is softer than the second material, the nonwoven of the first material (1) and the nonwoven of the second material being different to one another. The difference may include the type of bonding process used for the nonwovens, the polymers used for the fibers of the nonwovens; the density, basis weight, loft, thickness of the nonwovens used; the embossing pattern of the nonwovens used etc.

### Instructions for Testing

The combination herein may include user-instructions for testing, e.g., that explain to the user/purchaser how to test the test article (for example which part to feel for softness, or how to stretch to measure stretchability, or how to bend to measure flexibility, how to pull to measure fastening etc.), or how to compare the test article, in particular when a second material is provided on the test article, or on a second test article. In the latter case, the instruction may explain which two or more materials need to be tested, and what needs to be tested, and how the comparison may need to be performed, e.g., how to feel or bend or stretch both the first and second material simultaneously, or subsequently and compare respectively, softness, flexibility or stechability.

The instructions may be provided on the test article(s) or on the packaging material of the absorbent article(s). Alternatively, the user instructions may be provided on a separate item, which may be attached to the test article(s) or packaging material, or may be provided separately, for example attached to a shelf in store, as leaflet etc.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A consumer product comprising:
a package;
a disposable absorbent article adapted for use by infants, wherein the disposable absorbent article is contained within the package, the disposable absorbent article having an external surface and comprising a first material with a testable property; and
a first test article adapted for testing by a care-taker and for subsequent use by an infant, the first test article having an external surface comprising the first material with the testable property; and
wherein the external surface of the first test article is uncovered and accessible to a consumer.

2. The consumer product of claim 1, wherein the first test article is selected from the group consisting of: an article of clothing, a pillow, and a toy.

3. The consumer product of claim 1, wherein the first test article is defines a three-dimensional shape having an internal volume and comprising a resiliently compressible material.

4. The consumer product of claim 3, wherein the first test article is a toy, and the resiliently compressible material is selected from the group consisting of: a gas, a foam, cotton, and pulp.

5. The consumer product of claim 1, wherein the testable property is adapted to be testable by a user and by a care-taker by touch.

6. The consumer product of claim 1, wherein the testable property comprises a softness sensation and a stretchability of the first material.

7. The consumer product of claim 1, wherein the first material is selected from the group consisting of: a carded nonwoven material, a hydro-entangled nonwoven material, an embossed nonwoven material, and a calendar bonded nonwoven.

8. The consumer product of claim 1, further comprising instructions for instructing the care-taker how to test the first test article.

9. The consumer product of claim 1, further comprising a second test article comprising a second material having a testable property, and wherein the first test article further comprises the second material, and the first and second materials uncovered and accessible by a consumer to facilitate comparison of the first testable property and the second testable property.

10. The consumer product of claim 8, further comprising a display unit and wherein the disposable absorbent article is located within the display unit and the first test article and the second test article are attached to the display unit.
